(19) United States
(12) Patent Application Publication
(10) Pub. No.: US 2008/0221213 A1
(43) Pub. Date: Sep. 11, 2008

(54) Tablets Comprising a High Load of Strontium

(75) Inventor: Stephan Christgau, Gentofte (DK)

Correspondence Address: JONES DAY, 222 EAST 41ST ST, NEW YORK, NY, 10017, US

(73) Assignee: OSTEOLOGIX A/S SYMBION SCIENCE PARK, COPENHAGEN O, (DK)

(21) Appl. No.: 11/817,181

(22) PCT Filed: Nov. 7, 2005
(86) PCT No.: PCT/DK05/00710

§ 371 Date: Apr. 30, 2008

(30) Foreign Application Priority Data
Feb. 28, 2005 (DK) PCTDK2005000140
Jun. 17, 2005 (DK) PCTDK2005000401
Jun. 17, 2005 (DK) PCTDK2005000404

(51) Int. Cl. A61K 31/191

(52) U.S. Cl. 514/574

(57) ABSTRACT

Tablets comprising strontium in the form of a strontium salt such as strontium malonate. The tablets are designed to contain a very high load of the strontium salt (75% w/w or more), which is especially advantageous due to the fact that strontium must be administered in relative high amounts for therapeutic use. Furthermore, the tablets provided by the present invention lead to an improved bioavailability and presents a therapeutic advantage.

### FIELD OF THE INVENTION

The present invention relates to tablets comprising strontium in the form of a strontium salt such as strontium malonate. The tablets are designed to contain a very high load of the strontium salt, which is especially advantageous due to the fact that strontium must be administered in relative high amounts for therapeutic use. Furthermore, the tablets provided by the present invention lead to an improved bioavailability and presents a therapeutic advantage compared with the known product Protelos®.

### BACKGROUND OF THE INVENTION

In the previous years a number of publications and patent applications have emerged focusing on the advantages obtained in the therapy of e.g. a bone diseases and/or conditions resulting in a dysregulation of bone metabolism. However, the only product on the market is Protelos®, which is in the form of sachets, i.e. small paper bags containing a granulate of the active substance. The active substance is strontium ranelate. The Protelos® dosage form is intended to be suspended in a glass of water before ingestion, i.e. the patient in need thereof must swallow a relative large amount of water in order to obtain the necessary dose. It is generally recognized that such dosage forms lead to problems with respect to patient compliance because it is inconvenient to take and the taste and mouthfeel may not be acceptable.

Furthermore, the necessary daily dose of strontium ranelate is about 2 g, which is a very high dose that even if it is divided into two or more daily doses, gives problems with respect to e.g. formulation of tablets due to the resulting size of such tablets. Moreover, not all active therapeutically active substances posses the necessary properties with respect to flowability and compressibility that are requested in order to obtain a high load tablet.

Accordingly, there is a need for developing tablet dosage form containing a strontium salt that i) has a suitable size in order to allow the patient to easily swallow the tablet, ii) has a high load of strontium in order to minimize the administration frequency, and iii) that has suitable in vitro properties with respect to the release of the strontium salt from the tablet, and iv) has an in vivo behaviour that leads to the desired effect.

### SUMMARY OF THE INVENTION

In one aspect, the invention relates to a tablet comprising strontium malonate.

In some embodiments, the tablet contains 75% w/w or more, such as, e.g., 76% w/w or more, 77% w/w or more, 78% w/w or more, 79% w/w or more, 80% w/w or more, such as, e.g., 81% w/w or more, 82% w/w or more, 83% w/w or more, 84% w/w or more, 85% w/w or more of strontium malonate.

In certain embodiments, at least 65% w/w of the strontium malonate is released from the composition within 30 min. In particular embodiments, at least 65% w/w of the strontium malonate is released within 30 min, when tested in an in vitro dissolution test according to USP (paddle) and at 50 rpm. In some embodiments, the dissolution medium employed is an aqueous medium such as, e.g. 0.1 N HCl, water, or a buffer solution having a pH in a range corresponding to pH 3-10. In a specific embodiment, the medium is 0.1 N HCl. In certain embodiments of the tablet, at least 70% w/w such as, e.g., at least about 75% w/w, at least about 80% w/w, at least about 85% w/w, at least about 90% w/w or at least about 95% w/w is released within 30 min.

In some embodiments, the tablet provides a maximum plasma concentration of strontium from about 4 to about 24 hours after oral administration to a subject. In a specific embodiment, the tablet provides a maximum plasma concentration of strontium from about 4 to about 8 hours after oral administration to the subject. In another specific embodiment, the tablet provides a maximum plasma concentration of strontium from about 5.5 to about 24 hours after oral administration to the subject. In another specific embodiment, the tablet provides a maximum plasma concentration of strontium from about 5.5 to about 10 hours after oral administration to the subject.

In some embodiments of the tablet, the maximum plasma concentration of strontium is at least 1.5 times higher than the plasma concentration of strontium at 2 hours after oral administration of the tablet to a subject.

In certain embodiments of the tablet, the maximum plasma concentration of strontium is from about 0.5 mg/L to about 20 mg/L after oral administration of the tablet to a subject. For example, in a specific embodiment, the maximum plasma concentration of strontium is from about 1 mg/L to about 8 mg/L.

In some embodiments of the tablet, the tablet provides a steady state plasma concentration in a subject after repeated administration of the tablet to the subject through steady-state conditions, from about 5 mg/L to about 50 mg/L strontium. For example, in a specific embodiment, the steady state plasma concentration is from about 8 mg/L to about 20 mg/L strontium.

In certain embodiments of the tablet, the tablet has an improved bioavailability of strontium compared with that of PROTELOS® sachets containing strontium ranelate when orally administered in the same dose of strontium. In particular embodiments, the improved bioavailability expressed as AUC(tablet)/AUC(PROTELOS®, sachet) is larger than 1. For example, in specific embodiments, AUC(tablet)/AUC(PROTELOS®, sachet) is 1.05 or more such as, e.g., 1.1 or more.

In some embodiments of the tablet, the tablet is essentially bioequivalent with PROTELOS® when administered in a dose calculated as strontium that is at the about most about 90% w/w of the dose of strontium administered in the form of PROTELOS®. In specific embodiments, the bioequivalence is determined by means of at least one of the following parameters: tₘₐₓ, cₘₐₓ, AUC₀₋ₜ, AUC_{0-infinity}, W₅₀, and/or W₇₅.

In certain embodiments, the tablet further comprises a filler. In some embodiments the filler is microcrystalline cellulose. In a specific embodiment, the filler is present in a concentration of from about 5% w/w to about 15% w/w.

In some embodiments, the tablet further comprises a binder. In a specific embodiment, the binder is a polymeric compound. The polymeric compound can be, for instance, a polyvinylpyrrolidone. In some embodiments, the binder is present in a concentration of from about 1% w/w to about 8% w/w.

In certain embodiments, the tablet further comprises one or more lubricant(s). The lubricant can be, for instance, magnesium stearate and/or colloidal anhydrous silica. In some embodiments, the lubricant is present in a concentration from about 0.25% w/w to about 2.5% w/w

In a specific embodiment, the tablet has the following ingredients:

| | |
|---|---|
| strontium malonate | from about 75% w/w to about 90% w/w, |
| filler | from about 5% w/w to about 15% w/w, |
| binder | from about 1% w/w to about 8% w/w, and |
| lubricant(s) | from about 0.5% w/w to about 5% w/w. |

In another specific embodiment, the tablet has the following ingredients:

| | |
|---|---|
| strontium malonate | from about 75% w/w to about 90% w/w, |
| microcrystalline cellulose | from about 5% w/w to about 15% w/w, |
| Polyvidone | from about 1% w/w to about 5% w/w, |
| Colloidal anhydrous | from about 0.25% w/w to about 2.5% w/w, and |
| silica | |
| Magnesium stearate | from about 0.25% w/w to about 2.5% w/w. |

In another specific embodiment, the tablet has the following ingredients:

| | |
|---|---|
| strontium malonate | about 83.3% w/w, |
| microcrystalline cellulose | about 12% w/w, |
| Polyvidone | about 3.3% w/w, |
| Colloidal anhydrous silica | about 0.7% w/w, and |
| Magnesium stearate | about 0.7% w/w |

In some embodiments of the tablet, the amount of strontium malonate is from about 200 mg to 1800 mg such as, e.g., from about 300 mg to about 1500 mg, from about 500 mg to about 1500 mg or from about 600 mg to about 1200 mg. For instance, in certain embodiment of the tablet the amount of strontium malonate is from 900 mg to 1200 mg such as, e.g., from about 1050 mg to about 1150 mg. In some embodiments, the tablet is for once daily administration.

In some embodiments of the tablet, the amount of strontium malonate is from about 450 mg to about 600 mg such as, e.g., from about 500 mg to about 600 mg. In certain embodiments, the tablet is for twice daily administration.

### BRIEF DESCRIPTION OF THE DRAWINGS

FIG. 1 is a plot of the strontium plasma concentration vs. time following single dose oral administration of strontium malonate (0.6 g, 1.2 g and 2.4 g), strontium ranelate (2.0 g) and placebo compositions in human subjects.

FIG. 2 is a plot of the strontium plasma concentration vs. time for single and estimated repeated dose PK profiles for strontium malonate (1.2 g dose) from the single dose pharmacokinetic experiment described in Example 4.

### DETAILED DISCLOSURE OF THE INVENTION

Surprisingly, the present inventor have found that strontium malonate is a salt that has such advantageous properties with respect to flowability and compressibility that it is possible to manufacture tablets thereof with a load of 75% w/w or more. This mean that the present invention provides tablets that may have such a high content of strontium that a necessary daily dose can be provided in one or two tablets. Furthermore, the present invention fulfils a long-felt need by providing a very consumer acceptable dosage form that easily can be swallowed even if it contains a daily dose of strontium.

As mentioned above, the present invention relates to a tablet comprising a strontium salt preferable strontium malonate.

The tablets according to the invention have a high load of the strontium salt. Thus, the tablets contain 75% w/w or more, such as, e.g., 76% w/w or more, 77% w/w or more, 78% w/w or more, 79% w/w or more, 80% w/w or more, such as, e.g., 81% w/w or more, 82% w/w or more, 83% w/w or more, 84% w/w or more, 85% w/w or more of strontium malonate.

In order to enable a obtain a desired in vivo profile, the tablets must release the strontium salt in a suitable manner. The present inventors have found that a suitable in vivo plasma concentration—time profile is obtained when the tablets employed release at least 65% w/w of the strontium malonate from the composition within 30 min.

Accordingly, the strontium salt is released relatively fast from the tablets and this seems to be suitable in order to obtain a peak plasma concentration 4 hours or more after administration of the tablets (see the examples herein). Notably, the peak plasma concentration seems to occur 5.5 hours or more such as, e.g., 6 hours or more after administration. This means that the tablets conveniently can be administered at bed time in order to exert their effect in the morning. This is particularly suitable in relation to the morning stiffness related to certain bone conditions, i.e. administering a tablet at bed time provides an effective plasma concentration of strontium in the morning to alleviate the described symptoms.

More conveniently, a tablet according to the invention releases at least 65% w/w of the strontium malonate within 30 min, when tested in an in vitro dissolution test according to USP (paddle) and at 50 rpm.

To this end it should be emphasized that the dissolution medium for the in vitro dissolution test must be chosen taken the particular formulation technology of the tablet into consideration. This means that for plain tablets normally any traditionally used dissolution medium can be employed including an aqueous medium such as, e.g. 0.1 N HCl, water, or a buffer solution having a pH in a range corresponding to pH 3-10 such as, e.g., an acetate, citrate, phosphate or borate buffer solution. In a preferred aspect, the medium is 0.1 N HCl. However, if the tablets e.g. are designed to release the strontium salt in a delayed fashion, i.e. by providing the tablets with an enteric coat, a person skilled in the art will know how to adjust the dissolution test to these conditions using guidance from USP or similar official monographs. In such cases, the dissolution requirements mentioned above are fulfilled if the release is as described herein once the period of delay has ended.

A suitable release pattern of a tablet according to the invention provides a release of strontium malonate of at least 70% w/w such as, e.g., at least about 75% w/w, at least about 80% w/w, at least about 85% w/w, at least about 90% w/w or at least about 95% w/w within 30 min.

Apart from the requirements mentioned above, tablets according to the invention release at least about 80% w/w or more, such as, e.g., about 82% w/w or more, about 84% w/w or more, about 86% w/w or more, about 88% w/w or more, about 90% w/w or more, about 92% w/w or more, about 94% w/w or more, about 96% w/w or more, or about 98% w/w or more of the total amount of strontium malonate within about 45 minutes, such as, e.g. within about 40 minutes, within about 35 minutes, within about 30 minutes, within about 25 minutes or within about 20 minutes when tested in an in vitro dissolution test employing a suitable dissolution medium.

As mentioned above is it important that the tablets according to the invention provide a suitable plasma concentration vs time profile. As demonstrated in the examples herein, a tablet according to the invention provides a maximum plasma concentration of strontium from about 4 to about 24 hours such as, e.g., from about 4 to about 20 hours, from about 4 to about 15 hours, from about 4 to about 10 hours or from about 4 to about 8 hours after oral administration of a single dose to a subject.

In an important embodiment, the plasma concentration of strontium is provided from about 5.5 to about 24 hours such as, e.g., from about 5.5 to about 20 hours, from about 5.5 to about 15 hours, from about 5.5 to about 10 hours or from about 5.5 to about 8 hours after oral administration of a single dose to the subject.

Moreover, the maximum plasma concentration of strontium is at least 1.5 times higher than the plasma concentration of strontium at 2 hours after oral administration of the tablet to a subject.

In order to obtain the desired therapeutic effect and at the same time avoid too high plasma concentrations of strontium (to avoid unwanted side effects), the tablets according to the invention provides maximum plasma concentration of strontium that is from about 0.5 mg/L to about 20 mg/L such as, e.g., from about 1 mg/L to about 8 mg/L after oral administration of the tablet to a subject as a single dose.

At steady state concentrations are contemplated to be from about 5 mg/L to about 50 mg/L strontium such as, e.g., from about 8 mg/L to about 20 mg/L strontium after repeated oral administration of the tablets of the invention (see the examples herein). The frequency of the administration of the tablet may vary depending on, inter alia, the particular properties of the tablet. The tablet may be administered at least every 6 hours, such as every 12 hours, every 24 hours, every 48 hours, through steady state conditions. The tablet may be administered to the subject for at least ten consecutive days, such as at least ten to fourteen consecutive days, to obtain a steady state plasma concentration of strontium. As will be apparent to those of skill in the art, at steady state the plasma concentration of strontium will fluctuate over the course of the day. A person skilled in the art may realize that there may be other clinically acceptable ways of obtaining a steady state level of strontium plasma level, and that especially the administration of the pharmaceutical composition may be done in a number of alternative ways, such as twice or more daily.

Surprisingly, the present inventors have found that the tablets according to the invention improves the bioavailability of strontium compared with that obtained after oral administration of Protelos® sachets containing strontium ranelate in a dose that theoretically should be equivalent (same amount of strontium).

Accordingly, in a specific embodiment the invention relates to a tablet having an improved bioavailability of strontium compared with that of Protelos® sachets containing strontium ranelate when orally administered in the same dose of strontium.

The improved bioavailability can be expressed as the ratio between the Area Under the Curve, i.e. AUC(tablet)/AUC(Protelos®, sachet). An improved bioavailability is obtained when this ratio is larger than 1 such as, e.g., 1.05 or more, 1.1 or more, 1.2. or more or 1.3 or more.

As described below, other parameters may be used in order to determine an improved bioavailability.

The practical impact of an improved bioavailability is that a smaller dose of strontium can be administered in order to obtain an effect that is essentially bioequivalent with that of Protelos®, sachet. Accordingly, a specific embodiment of the invention relates to

a tablet containing strontium malonte, which tablet is essentially bioequivalent with Protelos® when administered in a dose calculated as strontium that is at the about most about 90% w/w of the dose of strontium administered in the form of Protelos®.

Parameters often used in bioequivalence studies are tₘₐₓ, Cₘₐₓ, AUC_{0-infinity}, AUC₀₋ₜ. Other relevant parameters may be W₅₀ and/or W₇₅. Accordingly, at least one of these parameters may be applied when determining whether bioequivalence is present. Furthermore, in the present context, two compositions are regarded as bioequivalent if value of the parameter used is within 80-125% of that of Protelos®.

In the present context “tₘₐₓ” denotes the time to reach the maximal plasma concentration (cₘₐₓ) after administration; AUC_{0-infinity} denotes the area under the plasma concentration versus time curve from time 0 to infinity; AUC₀₋ₜ denotes the area under the plasma concentration versus time curve from time 0 to time t; W₅₀ denotes the time where the plasma concentration is 50% or more of Cₘₐₓ; W₇₅ denotes the time where the plasma concentration is 75% or more Of Cₘₐₓ.

In the present context “steady state” means the state where one has achieved a stabile concentration of medicine throughout the day. Although one talks of a “stable” concentration in the blood, there will be variations or fluctuations through the day. The fluctuations will be influenced by the frequency of dosing of the drug as well as the elimination rate of the drug from circulation. However, the steady state is characterized by variations being on either side of the same centre point, and that the calculated mean concentration when calculated from a regular and frequent sampling over a time period from one drug administration to the next is a straight line. Normally the lowest blood levels are just before intake of the pharmaceutical composition, and the highest levels (Cₘₐₓ) obtained after a number of hours after intake of the pharmaceutical composition. Under steady state conditions, this variation should preferably be below 40% (understood as the difference between the lowest and highest plasma concentration expressed in % of the average levels observed over a 24 hour period in a subject given the strontium containing pharmaceutical composition). Physiologically the steady state is characterized by an overall equilibrium in the organism between the amount of drug being administered over a given period and the amount of drug metabolized and excreted over the same period.

As mentioned above, a tablet according to the present invention has a high load of the strontium salt. Accordingly, the amount of strontium malonate in the tablet is from about 200 mg to 1800 mg such as, e.g., from about 300 mg to about 1500 mg, from about 400 mg to about 1500 mg, from about 500 mg to about 1500 mg, from about 600 mg to about 1200 mg or from about 800 mg to about 1000 mg. Although the amount of strontium malonate is high, the tablets of the invention still has a suitable size for the patient to swallow it even if it contain the daily dose or half the daily dose.

In a specific embodiment, the amount of strontium malonate is from 900 mg to 1200 mg such as, e.g., from about 1050 mg to about 1150 mg. Such amounts are normally considered to be suitable for once daily administration.

In another embodiment, the amount of strontium malonate is from about 450 mg to about 600 mg such as, e.g., from about 500 mg to about 600 mg. Such amounts are normally considered to be suitable for twice daily administration.

In general, a tablet according to the invention may comprise 0.01 g, such as, e.g. at least about 0.025 g, at least about 0.050 g, at least about 0.075 g, at least about 0.1 g, at least about 0.2 g, at least about 0.3 g, at least about 0.4 g or at least about 0.5 g or from about 0.01 to about 2 g such as, e.g., from about 0.1 to about 2 g, from about 0.1 to about 1 g, from about 0.15 to about 0.5 g, from about 0.3 to about 2 g or from about 1 to about 2 g of ionic strontium.

It is envisaged that other strontium salts than strontium malonate may posses similar advantageous properties with respect to in vivo behavior (e.g. as expressed by an improved bioavailability and a suitable in vitro release pattern) and/or with respect to technical properties like flowability and/or compressibility (i.e. to obtain a tablet with a high load of strontium). Accordingly, the present invention is not construed to be limited to a specific strontium salt, but encompass strontium salts with similar advantageous properties as strontium malonate. Such strontium salts may be found among the following: strontium chloride, strontium chloride hexahydrate, strontium citrate, strontium succinate, strontium fumarate, strontium ascorbate, strontium aspartate in either L and/or D-form, strontium glutamate in either L- and/or D-form, strontium alpha-ketoglutarate, strontium pyruvate, strontium tartrate, strontium glutarate, strontium maleate, strontium methanesulfonate, strontium benzenesulfonate, strontium treonate, strontium ibuprofenate, strontium ascorbate and strontium salicylate.

A tablet according to the invention comprises one or more pharmaceutically acceptable excipients. In the following is given a description of suitable pharmaceutically acceptable excipients.

Pharmaceutically Acceptable Excipients

In the present context, the term “pharmaceutically acceptable excipient” is intended to denote any material, which is inert in the sense that it substantially does not have any therapeutic and/or prophylactic effect per se. A pharmaceutically acceptable excipient may be added to the active drug substance with the purpose of making it possible to obtain a pharmaceutical composition, which has acceptable technical properties. Although a pharmaceutically acceptable excipient may have some influence on the release of the active drug substance, materials useful for obtaining modified release are not included in this definition.

Excipients suitable for use in the present context include those normally used in formulation of solid dosage forms such as, e.g., fillers, binders, disintegrants, lubricants, flavouring agents, colouring agents, including sweeteners, pH adjusting agents, stabilizing agents, etc.

In a specific embodiment the tablet contains a filler.

Fillers/diluents/binders may be incorporated such as lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcema® P100, Emcocel®, Vivacel®, Ming Tai® and Solka-Floc®), hydroxypropylcellulose, L-hydroxpropylcellulose (low-substituted) (e.g. L-HPC-CH31, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium sulfate, calcium carbonate.

In tablets according to the present invention, especially microcrystalline cellulose, L-hydroxypropylcellulose, dextrins, maltodextrins, starches and modified starches have proved to be well suited. In a specific embodiment, the filler is microcrystalline cellulose.

Due to the high content of the strontium salt, the filler (and other pharmaceutically acceptable excipients) are present in a relativly low concentration. Thus, the filler is normally present in an amount of from about 5% w/w to about 15% w/w.

A tablet according to the invention may also contain a binder.

One or more binders may also be used. Suitable binders include those normally used within the pharmaceutical field including binders usually employed in wet granulation processes.

More specifically, examples include cellulose derivates including methylcellulose, hydroxypropylcellulose (HPC, L-HPC), hydroxypropylmethylcellulose (HPMC), microcrystalline cellulose (MCC), sodium carboxymethylcellulose (Na-CMC), etc.; mono-di-, oligo-, polysaccharides including dextrose, fructose, glucose, isomalt, lactose, maltose, sucrose, tagatose, trehalose, inulin and maltodextrin; gelatine, agar, alginic acid including sodium alginate, gummi arabicum, acaia; polyvinylpyrrolidone including Kollidon K30, Kollidon 90F or Kollidon VA64, and proteins including casein.

The type of binder employed depends on the formulation technique involved in the manufacturing of tablets. Thus, if e.g. a step of wet granulation is employed, binders like e.g. polyvinylpyrrolidone, maltodextrin, gelatine, agar etc. are suitable for use, whereas if direct compression is involved, binders like e.g. cellulose or cellulose derivatives are appropriate. A person skilled in the art will know how to select a suitable binder depending on the formulation technique, possibly with guidance from handbook like e.g. Remington's Pharmaceutical Science Handbook or Handbook of Pharmaceutical Excipients.

In a specific embodiment, the binder is a polymeric compound such as a polyvinylpyrrolidone.

Normally, the binder is present in a concentration of from about 1% w/w to about 8% w/w.

A disintegrant may also be included in the tablet. Typically, a disintegrant is selected from the group consisting of: croscarmellose sodium (a cross-linked polymer of carboxymethylcellulose sodium), crospovidone, starch NF; polacrilin sodium or potassium and sodium starch glycolate. Those skilled in the art will appreciate that it is desirable for tablets to disintegrate within 30 minutes such as within 10 min. Therefore, if present, the disintegrant used preferably results in the disintegration of the tablet within 30 minutes, more preferable within 10 min.

Examples of disintegrants that may be used are e.g. cellulose derivatives, including microcrystalline cellulose, low-substituted hydroxypropyl cellulose (e.g. LH 22, LH 21, LH 20, LH 32, LH 31, LH30); starches, including potato starch; croscarmellose sodium (i.e. cross-linked carboxymethylcellulose sodium salt; e.g. Ac-Di-Sol®); alginic acid or alginates; insoluble polyvinylpyrrolidone (e.g. Polyvidon® CL, Polyvidon® CL-M, Kollidon® CL, Polyplasdone® XL, Polyplasdone® XL-10); sodium carboxymethyl starch (e.g. Primogel® and Explotab®).

Moreover, a tablet according to the invention may comprise one or more glidants or lubricant(s).

Glidants and lubricants may be incorporated such as stearic acid, metallic stearates including magnesium stearate, talc, waxes and glycerides with high melting temperatures, colloidal silica, sodium stearyl fumarate, polyethylenglycols and alkyl sulphates.

In a specific embodiment, the lubricant is magnesium stearate and/or colloidal anhydrous silica.

The lubricant is normally present in a concentration from about 0.25% w/w to about 2.5% w/w

In the following are given specific embodiments of the present invention.

In a preferred embodiment, a tablet according to the invention has the following ingredients:

| | |
|---|---|
| strontium malonate | from about 75% w/w to about 90% w/w |
| filler | from about 5% w/w to about 15% w/w |
| binder | from about 1% w/w to about 8% w/w |
| lubricant(s) | from about 0.5% w/w to about 5% w/w |

More specifically, a tablet according to the present invention has the following composition:

| | |
|---|---|
| strontium malonate | from about 75% w/w to about 90% w/w |
| microcrystalline cellulose | from about 5% w/w to about 15% w/w |
| Polyvidone | from about 1% w/w to about 5% w/w |
| Colloidal anhydrous silica | from about 0.25% w/w to about 2.5% w/w |
| Magnesium stearate | from about 0.25% w/w to about 2.5% w/w |

The tablets used in the clinical trials have the following composition that, accordingly, is of specific interest as a separate embodiment of the invention:

| | |
|---|---|
| strontium malonate | about 83.3% w/w |
| microcrystalline cellulose | about 12% w/w |
| Polyvidone | about 3.3% w/w |
| Colloidal anhydrous silica | about 0.7% w/w |
| Magnesium stearate | about 0.7% w/w |

Specific compositions appear from the examples herein.

Other appropriate pharmaceutically acceptable excipients may include colorants, flavouring agents, and buffering agents.

Furthermore, the tablets of the invention may be provided with a suitable coating such as e.g. a film coating.

### EXAMPLES

### Example 1

### Preparation of 300 mg Strontium Malonate Tablets

The following manufacturing procedure was followed for the preparation of approximately 10,000 tablets containing 300 mg of strontium malonate (anhydrous—may be prepared as described below) and other ingredients in the proportions specified in the table below.

### Pharmaceutical Composition Containing 300 mg Strontium Malonate

### Tablet Formulation

| Ingredient | Amount (mg)/tablet |
|---|---|
| Strontium malonate (anhydrous) | 300 mg |
| Microcrystalline Cellulose Ph. Eur. | 43.5 mg |
| Polyvidone Ph. Eur. | 12 mg |
| Colloidal anhydrous silica Ph. Eur. | 2.5 mg |
| Magnesium Stearate Ph. Eur. | 2.5 mg |
| Purified water Ph. Eur. | q.s. |

Strontium malonate (3600 g) and Microcrystalline Cellulose (Avicell, 180 g) were mixed thoroughly in suitable mixing equipment. After mixing, the material was filtered through a 1 mm diameter sieve. Over a period of 2 minutes and under constant mixing, Polyvidone (144 g) and purified water (450 g) were added to the mixture. When a homogenous granulate was obtained, it was sifted through a 1.2 mm sieve and placed on trays for drying, and the trays placed in a drying cupboard at 40° C., for 2½ to 3 hours. Drying was monitored by measuring the relative humidity of the air in the drying cupboard, and drying was considered complete when the water content was between 25-40% RH. The dried granulate was passed through a 1 mm diameter sieve. Colloidal Anhydrous Silica (23 g) and remaining Microcrystalline Cellulose (Avicell, 284 g) were mixed thoroughly and sieved through a 0.7 mm diameter sieve. The granulate and the silica-cellulose mixture were blended. Magnesium Stearate (23 g) was sieved through a 0.7 mm diameter sieve and premixed with approximately 350 g of the mixture, and when a homogenous mixture was obtained, the rest of the mixture was added. The mixture was added to a compression tabletting machine, and 360 mg (containing 300 mg strontium malonate) tablets were pressed in 8 mm cylindrical tablet stamps.

### Manufacture of Anhydrous Strontium Malonate at 95-100° C.

The present example is provided to illustrate the applicability of a high temperature synthetic method to produce strontium malonate in kg-scale quantities.

63.2 kg of purified water was mixed with 15.75 kg of strontium hydroxide octahydrate (Riedel de Hahn, Germany) in a closed 100 L commercial scale reactor under a nitrogen atmosphere. The reactor was made of glass-lined stainless steel providing a chemically inert surface that is easily cleaned after completion of manufacture. The strontium hydroxide was dissolved at a temperature of 97.1° C., and the resulting unclear solution was transferred to a nitrogen filled pressure filter (heated 96° C.) to remove strontium carbonate. The reactor was cleaned with diluted hydrochloric acid, tap water and finally with purified water.

A 10 L polyethylene (PE) container was charged with 5.630 kg (54.1 mol) of malonic acid (Sigma, USA) and purified water (4.1 kg), and the mixture was shaken until a clear solution was obtained. The malonic acid solution was then filtered into the 100 L reactor containing the strontium hydroxide solution. This reactor was of similar design to the one used for dissolving the strontium hydroxide as described above. The PE container and the filter were washed with purified water (1.4 L) for future use. The reactor was evacuated and equilibrated with nitrogen three times before the solution was heated to a gentle reflux under a nitrogen atmosphere.

The hot strontium hydroxide solution was filtered and added into the reactor containing the strontium malonate solution at a starting temperature of 96.2° C., and the resulting mixture was continually mixed for a period of 21 minutes. At the end of the mixing period the reaction temperature was 97.9° C. After 2 hours and 51 minutes, the reaction temperature was 44.4° C. The reaction product was filtered on a pressure filter preheated to 48° C. The filter cake containing the strontium malonate was washed with purified water (13.2 kg) preheated to 45.1° C., and subsequently dried in a vacuum at 70° C. to give 9.631 kg of anhydrous strontium malonate, which corresponds to 93.9% of the theoretical yield.

### Example 2

### Preparation of 600 mg Strontium Malonate Tablets

The following manufacturing procedure may be used for the preparation of approximately 5000 tablets containing 600 mg of strontium malonate (anhydrous—may be prepared as described above) and other ingredients in the proportions specified in the table below.

### Pharmaceutical Composition Containing 600 mg Strontium Malonate

### Tablet Formulation

| Ingredient | Amount (mg)/tablet |
|---|---|
| Strontium malonate (anhydrous) | 600 mg |
| Microcrystalline Cellulose Ph. Eur. | 87 mg |
| Polyvidone Ph. Eur. | 24 mg |
| Colloidal anhydrous silica Ph. Eur. | 5 mg |
| Magnesium Stearate Ph. Eur. | 5 mg |
| Purified water Ph. Eur. | q.s. |

Strontium malonate (3600 g) and Microcrystalline Cellulose (Avicell, 180 g) are mixed thoroughly in suitable mixing equipment. After mixing the material is filtered through a 1 mm diameter sieve. Over a period of 2 min and under constant mixing, Polyvidone (144 g) and purified water (450 g) are added to the mixture. Additional water may be added if required for obtaining a homogenous granulate. When a homogenous granulate has been obtained, it is sifted through a 1.2 mm sieve and placed on trays for drying, and the trays are placed in a drying cupboard at 40° C., for 2½-3 hours. Drying is considered complete when the water-content was between 25-40% RH. The dried granulate is passed through a 1 mm diameter sieve. Colloidal Anhydrous Silica (23 g) and remaining Microcrystalline Cellulose (Avicell, 284 g) are mixed thoroughly and sieved through a 0.7 mm diameter sieve. The granulate and the silica-cellulose mixture are blended. Magnesium Stearate (23 g) is sieved through a 0.7 mm diameter sieve and premixed with approximately 350 g of the mixture, and when a homogenous mixture has been obtained, the rest of the mixture is added. The mixture is added to a compression tabletting machine, and 721 mg (containing 600 mg strontium malonate) tablets are pressed in cylindrical oblong tablet stamps. The above-described manufacturing procedure is readily scaled up for preparation of larger batches of tablets.

Skilled artisans will recognize that the tablet size may be varied by varying the tablet stamping tools. Furthermore, variations in the strength of the tablet can be readily accommodated by modifications of the above-described compositions.

### Example 3

### Dissolution and Disintegration Testing of 300 mg Strontium Malonate Tablets

Both dissolution and disintegration time of the 300 mg strontium malonate tablet manufactured as described in Example 1 were determined. This example provides a description of the analytical procedures used for these analysis as well as representative results from analysis of two batches of tablets.

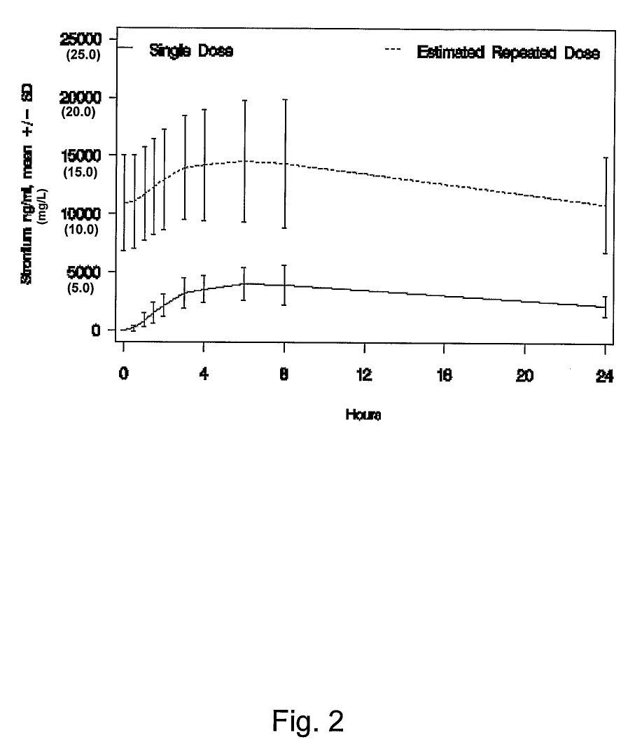

The tablets manufactured as described in Example 1 were subjected to dissolution testing according the U.S. Pharmacopoeia (USP) method 28 in a paddle apparatus “2” as specified in the pharmacopoeia (see also the European pharmacopoeia, 5^{th} edition figure 2.9.3.1). 900 ml (±1%) 0.1 N HCl is added to the chamber. The paddle was operated at 50 RPM and the temperature adjusted to and maintained at 37±0.5° C. during the dissolution experiment. One tablet was placed in the dissolution chamber according to the Pharmacopoeia. After 30 minutes, a sample was taken of the dissolution medium from the middle of the dissolution chamber. Strontium content (as free Sr²⁺ ions) in the sample of the dissolution medium was performed by means of Inductively-Coupled Plasma-Mass Spectrometry (ICP-MS) on a Perkin Elmer instrument Elan 6100. Samples were received in 10 mL tubes (Nunc), and the whole sample was transferred to a 2000 mL measuring bottle with distilled-water. Further dilution with 0.5% HNO₃ was performed. 10 mL of the sample was added to internal standards (Gallium, Ga, present in the two naturally occurring isotopes ⁶⁹Ga and ⁷¹GA) prior to the ICP-MS analysis. The analysis of the sample on the ICP-MS apparatus was performed according the manufacturer's instructions.

Calibration was performed using a dilution of a certified reference standard from Merck (Sr 1000 mg/ml). A certified control material “TM-DWS” (a trace-element fortified reference sample derived from filtered sea-water sample from Lake Ontario provided by the Canadian National Water Research Institute) was used as control sample and is analyzed an appropriate number of times in the analytical sequence. Furthermore, a certified reference standard from Baker was used as a calibration control sample.

The ICP-MS instrument measured the intensity of ⁸⁶Sr, ⁸⁸Sr and Ga as net intensities (counts per second) in all solutions and the concentration in μg/L was calculated by linear regression from analysis of the calibration curve by use of internal standard method. Calibration range was 1-200 μg/L. Samples above the calibration range were reanalyzed after additional dilutions to fit the calibration range.

### ICP-MS Measurement

The strontium concentration in the samples from each dissolution chamber was analyzed and the result expressed as the percent strontium in relation to the total strontium content in the tablet. Table 1 shows representative results from analysis of two batches of 300 mg strontium malonate tablets produced as described in Example 1.

TABLE 1

| Dissolution test | | Dissolution test | |
|---|---|---|---|
| Experimental | | Experimental | |
| run | Result | run | Result |
| 1 | 99% | 1 | 87% |
| 2 | 83% | 2 | 98% |
| 3 | 87% | 3 | 94% |
| 4 | 98% | 4 | 92% |
| 5 | 90% | 5 | 71% |
| 6 | 92% | 6 | 82% |
| Average | 91.5% (SD 6.2%) | Average | 87.3% (SD 9.8%) |

Another important analysis of the tablets manufactured as described in Example 1 is determination of disintegration time. This analysis was performed essentially as described in the European Pharmacopoeia 5^{th} ed. Section 2.9.1, test A. Briefly described the test was performed by placing 800 mL deionized water in the container where after the container was placed in a thermostatted water bath maintained at 37±2° C. One tablet was placed in each of the six chambers in the apparatus. The chambers were placed in the container so that there is at least 25 mm free space over the bottom of the container and at least 5 mm from the top of the chambers to the surface of the disintegration medium (deionized water). When the chambers with tablets were placed in the dissolution medium, a stop-watch is started and the tablets were monitored visually. The time until the last of the 6 tablets have disappeared was noted and this constituted the disintegration time. A tablet was considered to be disintegrated when there was no more visible traces of the tablet in the reaction chamber and/or when remnants in the chamber is devoid of any regular hard or shaped pieces. Table 2 below shows the results of disintegration testing of two batches of 300 mg strontium malonate tablets manufactured as described in Example 1.

TABLE 2

| | |
|---|---|
| 300 mg strontium | 300 mg strontium |
| malonate | malonate |
| Tablet batch 1 | Tablet batch 2 |

| | | | |
|---|---|---|---|
| Disintegration test | 8 min | Disintegration test | 6 min |

### Results

It is apparent the 300 mg strontium malonate tablets manufactured as described in Example 1 were able to disintegrate rapidly, with a disintegration time as determined according to current European pharmacopoeia methods of less than 10 min for two representative batches. The dissolution testing of the tablets from the same two batches was determined in 6 independent analytical runs for tablets from the same two batches. The average dissolution results showed a recovery of 91.5% and 87.3% after 30 min dissolution performed according to the U.S. Pharmacopoeia.

Combined these two results show that the 300 mg strontium malonate tablets have the ability to rapidly disintegrate and dissolve under analytical conditions designed to provide a relevant representation of the condition in the stomach after oral intake in humans. Accordingly it can be concluded that the pharmaceutical composition described in Example 1 is able to provide a rapid and reliable release of strontium malonate.

### Example 4

### Determination of Pharmacokinetic Properties of Strontium Malonate

The pharmacokinetic properties of the 300 mg strontium malonate tablet prepared in Example 1 were determined in a single oral dose pharmacokinetic study in humans. To enable accurate assessment of pharmacokinetic properties of the strontium malonate-containing tablets of Example 1, three dose-levels were administered to the study subjects, a 0.6 g dose of strontium malonate (2 tablets), a 1.2 g dose of strontium malonate (4 tablets) and a 2.4 g dose of strontium malonate (8 tablets). Furthermore, 2.0 g of strontium ranelate was administered from a sachet dosage form (Protelos®, Servier Laboratories Ltd.) to a separate treatment group to do a comparison of pharmacokinetic properties between the dosages of strontium malonate and strontium ranelate. An additional goal of the study was to determine the bioequivalent dose of the tablets of Example 1, containing strontium malonate, giving the same amount of absorbed strontium as the 2.0 g strontium ranelate dose present in a commercially available sachet formulated dosage form.

Placebo tablets were used in the study. These tablets were manufactured as described in Example 1, but all strontium malonate was substituted with microcrystalline cellulose (avicell). The placebo tablets were stamped in the same tablet machine as the strontium malonate tablets and had the same visual appearance.

The percentages of strontium and molecular weights of the particular salt forms of strontium malonate and strontium ranelate used to calculate the theoretical bioequivalent dose of strontium malonate to 2.0 g strontium ranelate is shown below in Table 3.

TABLE 3

| | Dose | | | | |
|---|---|---|---|---|---|
| Group | (mg) | Strontium salt | MW | % Sr | Dose Equivalent |
| 1 | 1200 | Sr-malonate | 189.6 | 46.2 | 1200 mg = 554 mg |
| | | (*0H₂O) | | | Sr⁺⁺*⁾ |
| 2 | 2000 | Sr-ranelate | 639.6 | 27.4 | 2000 mg = 548 mg |
| | | (*7H₂O) | | | Sr⁺⁺ |

In the study, sixty healthy male volunteers, between 18 and 40 years of age were enrolled in the pharmacokinetic study. Prior to initiation of the study, the study protocol was approved by an Independent Ethics Committee, and all procedures were performed according to Good Clinical Practice.

### Trial Objectives and Purpose

The primary objective of the study was to obtain pharmacokinetic (PK) data (AUC_{5 weeks} and Cₘₐₓ) on the strontium malonate tablets prepared according to Example 1. Secondary objectives of the study were:

to demonstrate bio-equivalence of 2.0 g of strontium ranelate and the medium dose of 1.2 g of strontium malonate;

to estimate the dose of strontium malonate that is equivalent to 2.0 g of strontium ranelate, in terms of AUC;

to obtain data on safety parameters of strontium malonate;

to obtain data on markers of calcium balance; and

to obtain data on biochemical markers of bone turnover.

As used herein, bioequivalence (BE) is defined as follows: Two medicinal products are bioequivalent if they are pharmaceutical equivalents or pharmaceutical alternatives, and if their bioavailabilities after administration in the same molar dose are similar to such degree that their effects, with respect to both efficacy and safety, will be essentially the same (CPMC/EWP/QWP/1401/98).

In the present study, BE is assessed by AUC and Cₘₐₓ which were both evaluated during the 5 week follow up period on the strontium malonate medium dose (1.2 g) and 2.0 g strontium ranelate (Protelos®). BE is demonstrated if confidence intervals of the ratio of the two interventions are lying within the 80-125% range for both AUC and Cₘₐₓ.

The study was performed as a semi-blinded randomized, single dose, parallel-group study, with 12 healthy volunteers enrolled for each of the 5 administration regimens.

Subjects were allocated to one of the strontium malonate arms or the placebo arm received a total of 8 identical tablets. Either:

0.6 g dose-group: 2 strontium malonate (SM) 300 mg tablets and 6 placebo tablets,

1.2 g dose-group: 4 strontium malonate (SM) 300 mg tablets and 4 placebo tablets,

2.4 g dose-group: 8 strontium malonate (SM) 300 mg tablets or

Placebo: 8 placebo tablets.

An additional study group was administered strontium ranelate (Protelos®). As Protelos® is formulated as yellowish granules for suspension in water it was decided, for pharmaceutical reasons, not to include Protelos® in the blinding.

For practical reasons, the treatment and follow up of all 60 subjects were divided in 6 separate treatment “occasions”, where 10 subjects were included and treated per occasion. The subjects were randomized in blocks of 10 to one of the 5 interventions at baseline; 2 subjects for each intervention. All subjects, including the placebo group, were asked to take the study medication in a glass of 200 ml water.

Each intervention was performed as a single oral dose administration in the evening, at least six hours after the last meal, i.e., the study subjects were not to eat dinner the evening of the investigational medicinal product administration.

The study drug administration took place at 7 p.m. During the 24 h study period following the administration of the study medication, the subjects were given a meal at 8 a.m. and 12 a.m. and a light snack at 3 p.m.

For determination of pharmacokinetic properties of the strontium compounds the main analysis method consisted of determination of ionic strontium in serum samples from the study subjects. Blood samples were taken at regular intervals after administration of the study drug, and processed to serum for determination of strontium content by ICP-MS (inductively coupled plasma mass spectroscopy). Blood samples were withdrawn from subjects at 0 minutes, 30 minutes, 60 minutes, 90 minutes, 2 hours, 3 hours, 4 hours, 6 hours, 8 hours, 24 hours, 1 week, 3 weeks and 5 weeks after administration of the study drug.

Determination of strontium as Sr⁺⁺, in serum was performed using ICP-MS for quantitative determination of ionic strontium concentration in the formulation. Prior to analysis the formulation was diluted in 0.1 M HNO₃ before measurement, to obtain a suitable concentration range of the solution. A Perkin Elmer Elan 5000 system equipped with a cross-flow nebulizer was used for the ICP-MS measurements.

### Results

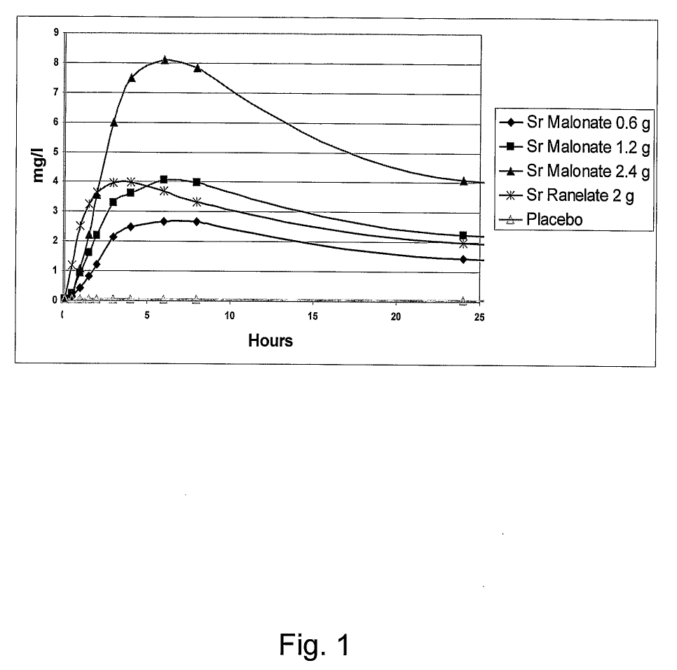

The study medications were well tolerated in all 60 study subjects and all subjects were available for the full study period. Two subjects did not have 5 week samples available and thus, the sample cohort for the per protocol (PP) analysis were 58 compared with 60 for the full intention to treat analysis (ITT). All subjects had a well defined strontium profile curve enabling a calculation of the AUC_{5 week} (see FIG. 1). Some subjects reported mild symptoms, during the first 24 hour period (headache, cold-symptoms, allergies, etc.), none of which were serious or likely to be related to the study medication, and the reports of overall symptoms were evenly distributed in all treatment groups. However of note, 2 of the 12 subjects given strontium ranelate reported diarrhea, whereas this was not seen in any of the 36 subjects treated with strontium malonate, indicating that the strontium malonate may be devoid of the diarrhea side effect which is associated with strontium ranelate.

The pharmacokinetic properties in the three study groups treated with strontium malonate demonstrated a clear dose response in the uptake of strontium from the administered preparation of the product (FIG. 1). AUC was calculated in arbitrary units in both the ITT and the PP population (Tables 4 and 5). It is apparent from the PK graphs of circulating strontium levels depicted in FIG. 1, and from the tabular presentation in Table 4 of AUC_{5 week} data for each of the four treatment groups that 1.2 g strontium malonate shows slightly higher bioavailability than the 2.0 g of strontium ranelate. The theoretical bioequivalent dose to 2 g strontium ranelate (Protelos™), was found to be 1.09 g by inverse regression. This was performed by using the following model for assessing the linear relationship between strontium levels administered to the study subjects (dose) and the resulting uptake of strontiumin to circulation assessed as AUC_{5 week}:

AUC=α·Dose^{β} [symbol] log(AUC)=log(α)+β·log(Dose)

The dose of strontium malonate, which in terms of AUC was bio-equivalent with 2.0 g Protelos® was estimated from the dose-response relationship established in the model above, by inverse regression. The strontium malonate estimate was based on the actual estimates of α and β obtained when the values from the groups of study subjects treated with 0.6, 1.2 and 2.4 g strontium malonate was applied in the model. Using the AUC value obtained in the group treated with the 2.0 g strontium ranelate sachet formulation the bioequivalent strontium malonate dose was found to 1.09 g and a 95% confidence intervals was calculated (Fieller's theorem) to [1.073, 1.115].

Thus, the study demonstrated a significant difference in the bioavailability of strontium malonate tablets of Example 1 compared to the strontium ranelate dosage form, as similar strontium concentrations were obtained after administration of 2.0 g strontium ranelate could be obtained with a strontium malonate dose significantly below the equimolar dose of 1.2 g.

TABLE 4
Analysis of AUC - 5 Weeks (Intention to Treat Analysis, ITT)
(SM = strontium malonate)

| | | | |
|---|---|---|---|
| AUC Estimate (SE) | 90% CI | Wald test | P-value |

| | |
|---|---|
| SM 0.6 g | 275273 (24317) |
| SM 1.2 g | 386901 (39455) |
| SM 2.4 g | 654617 (63742) |
| Protelos ® | 356347 (24434) |
| 2.0 g | |
| Placebo | 40992 (1984) |

Ratio (SM/Protelos ®)

| | | | | |
|---|---|---|---|---|
| SM 0.6 g | 0.77 (0.086) | [0.63, 0.92] | 6.94 | 0.008 |
| SM 1.2 g | 1.09 (0.133) | [0.87, 1.31] | 0.41 | 0.52 |
| SM 2.4 g | 1.84 (0.219) | [1.48, 2.20] | 14.6 | 0.0001 |

TABLE 5
Analysis of AUC - 5 Weeks (SM = strontium malonate)
(Per protocol analysis, PP)

| | | | |
|---|---|---|---|
| AUC Estimate (SE) | 90% CI | Wald test | P-value |

| | |
|---|---|
| SM 0.6 g | 276048 (26625) |
| SM 1.2 g | 412109 (33249) |
| SM 2.4 g | 654617 (63742) |
| Protelos ® | 356347 (24434) |
| 2.0 g | |
| Placebo | 40992 (1984) |

Ratio (strontium malonate/Protelos ®)

| | | | | |
|---|---|---|---|---|
| SM 0.6 g | 0.77 (0.092) | [0.62, 0.93] | 6.04 | 0.014 |
| SM 1.2 g | 1.16 (0.122) | [0.96, 1.36] | 1.63 | 0.201 |
| SM 2.4 g | 1.84 (0.219) | [1.48, 2.20] | 14.6 | 0.0001 |

For Tₘₐₓ, it was observed that the tablets of Example 1 (containing strontium malonate) resulted in a delayed uptake compared to strontium ranelate. Tₘₐₓ was 1-2 hours later in all three strontium malonate-treated groups compared to the strontium ranelate-treated groups.

### Conclusions

The single dose pharmacokinetic study demonstrated that strontium was taken up in a dose dependent fashion when strontium malonate was administered as an oral tablet formulated pharmaceutical product to human subjects. The bioavailability of strontium from the tablets of Example 1 was better than from the strontium ranelate dosage form (Protelos®), as the bioequivalent dose of strontium malonate to 2.0 g strontium ranelate is 1.09 g, significantly less than the theoretically equimolar dose of 1.2 g of the composition of Example 1.

### Example 5

### Calculation of Steady State Plasma Levels from Single Dose Pharmacokinetic Experiment in Man

The modelling of the steady state level of circulating strontium was based on individual 0 hours to 5 weeks single dose strontium profiles as obtained from the single dose pharmacokinetic experiment described above in Example 4. From these profiles individual estimated 0-24 hours steady state profiles was calculated as the sum of

a. the sample values at 0, 0.5, 1, 1.5, 2, 3, 4, 6, 8, and 24 hours on day 1 (data recorded directly in the trial); and
b. corresponding terms of e.g., 1 hours on day 2, 3, 4 etc. The sum of these terms is found as the sum of an infinite series using a model for the terminal elimination. This corresponds to using the accumulation factor (1).

This modelling yields estimates of the steady state strontium levels with two assumptions:

1. Dose linearity of strontium pharmacokinetics within the range of concentrations involved/
2. The model for the terminal elimination is reasonably accurate.

The terminal elimination was estimated for each subject on basis of the 24 hours and the 1, 2, and 3 weeks concentrations. The one-compartment model with exponentially decreasing concentrations was used, partly because a reasonable fit was observed and partly because a more complex model could only be estimated on the available data with great uncertainty.

With t being a time point within the first 24 hours the formal estimation of the steady state concentration will be given according to the formula listed below:

Cₛₛ(t)=C(t)+Ĉ(t+24)+Ĉ(t+48)+Ĉ(t+72)+ . . . .

where C(t) is the measured concentration after single dose administration, and Ĉ(t+i·24) are estimated from the single dose terminal elimination model. Assuming the one-compartment model this reduces to

$${C}_{ss} ( t ) = C ( t ) + \frac{K exp ( - {k}_{e} t )}{1 - exp ( - {k}_{e} 24 )}$$

with kₑ being the elimination constant and K being the proportionality constant in the terminal elimination. The parameters in the one-compartment model were estimated by linear regression on the log-transformed concentration values.

All calculations were based on baseline adjusted strontium concentrations, as the strontium concentrations in the baseline situation is not zero due to the intake of small amounts of strontium in the normal diet of the study subjects.

### Results

A total of six concentration values were marginally below zero after baseline adjustment. These observations were set to 1 ng/ml to avoid problems with log-transformation. By inspecting log concentration plots (not shown) it was detected that the elimination of strontium is not well described by a one-compartment model from 24 hours and onwards. It is however assessed that for summing to infinity the one-compartment model will give reasonable estimates in this situation where a single strontium dose has been administered to the study subjects.

The observed 24 hours single dose concentrations are shown together with the estimated steady state steady concentrations after repeated oral dosing in FIG. 2.

The estimated mean steady state concentration ranges approximately from 11000 ng/ml to 14000 ng/ml during the time from dosing at time=0 to time=24 hours. The plot shown in FIG. 2 is based on arithmetic means and standard deviations.

The estimated mean Cₘₐₓ steady state values are shown in table 6 calculated for the four strontium treated groups of the single dose pharmacokinetic study described in Example 4. Means and confidence intervals are calculated on the log-scale and converted back to the original scale for presentation.

TABLE 6

| | Estimated Steady | | | Estimated Steady | | |
|---|---|---|---|---|---|---|
| | State Cmax | | | State Cmax | | |
| | (ng/mL) | | | (mg/L) | | |
| Treatment | Estimate | Lower CI | Upper CI | Estimate | Lower CI | Upper CI |
| strontium | 9561 | 7535 | 12133 | 9.561 | 7.535 | 12.133 |
| malonate 0.6 g | | | | | | |
| strontium | 14019 | 11048 | 17790 | 14.019 | 11.048 | 17.790 |
| malonate 1.2 g | | | | | | |
| strontium | 23845 | 18791 | 30258 | 23.845 | 18.791 | 30.258 |
| malonate 2.4 g | | | | | | |
| Protelos 2.0 g | 12698 | 10006 | 16113 | 12.698 | 10.006 | 16.113 |

The dose response was evaluated and concluded linear with respect to Cₘₐₓ. This covered the concentration range (not baseline corrected) from 2.947 to 8.697 mg/L as assessed by the mean strontium Cₘₐₓ values, and individual Cₘₐₓ values from 1.027 to 16.510 mg/L. The estimated concentration values for the strontium malonate 1.2 g dose in table 6 are within these ranges and it may be assumed that the dose linearity applies. Further, it may be concluded that the assumptions which the estimation of the steady state concentrations were based on are fulfilled up to the concentration level resulting from a strontium malonate 1.2 g dose. It is therefore finally concluded that daily oral dosing with 1.2 g strontium malonate formulated in tablets, such as the tablet formulation described in Example 1, and used in the pharmacokinetic study as described in Example 4 will lead to a maximum steady state concentration of mean 14.109 mg/L and CI from 11.048 to 17.790 mg/L.

### Example 6

### Composition Comprising a Combination of a Strontium Containing Compound and 6-(5-carboxy-methyl-hexyloxy)-2,2-dimethyl-hexanoic acid

The following example tend to illustrate that strontium malonate may be formulated together with another active substance in a tablet according to the invention.

TABLET FORMULATION

| Ingredient | Amount (mg) |
|---|---|
| 6-(5-carboxy-methyl-hexyloxy)-2,2-dimethyl- | 20 mg |
| hexanoic acid, strontium salt | |
| strontium malonate | 520 mg |
| Lactose | 20 mg |
| Corn starch (for mixing) | 15 mg |
| Corn starch (for paste) | 15 mg |
| Magnesium Stearate (1%) | 10 mg |
| Total | 600 mg |

6-(5-carboxy methyl-hexyloxy)-2,2-dimethyl-hexanoic acid, strontium salt and strontium malonate, lactose and cornstarch (for mixing) is blended to uniformity. The cornstarch for paste is suspended in 200 ml of water and heated with stirring to form a paste. The paste is used to granulate the mixed powders (wet granulation). The wet granules are passed through a number 8 hand screen and dried at 80° C. After drying, the granules are lubricated with 1% magnesium stearate and pressed into a tablet. Such tablets can be administered to a human subject in need thereof from one to two times daily.

1. A tablet comprising strontium malonate and microcrystalline cellulose.

2. The tablet according to claim 1, containing 75% w/w or more of strontium malonate.

3. The tablet according to claim 1, wherein at least 65% w/w of the strontium malonate is released from the composition within 30 min.

4. The tablet according to claim 1, wherein at least 65% w/w of the strontium malonate is released within 30 min, when tested in an in vitro dissolution test according to USP (paddle) and at 50 rpm.

5. The tablet according to claim 4, wherein the dissolution medium employed is an aqueous medium.

6. The tablet according to claim 5, wherein the medium is 0.1 N HCl.

7. The tablet according to claim 3, wherein at least 70% w/w is released within 30 min.

8. The tablet according to claim 1, providing a maximum plasma concentration of strontium from about 4 to about 24 hours after oral administration to a subject.

9. The tablet according to claim 8, providing a maximum plasma concentration of strontium from about 4 to about 8 hours after oral administration to the subject.

10. The tablet according to claim 8, providing a maximum plasma concentration of strontium from about 5.5 to about 24 hours after oral administration to the subject.

11. The tablet according to claim 10, providing a maximum plasma concentration of strontium from about 5.5 to about 10 hours after oral administration to the subject.

12. The tablet according to claim 1, wherein the maximum plasma concentration of strontium is at least 1.5 times higher than the plasma concentration of strontium at 2 hours after oral administration of the tablet to a subject.

13. The tablet according to claim 1, wherein the maximum plasma concentration of strontium is from about 0.5 mg/L to about 20 mg/L after oral administration of the tablet to a subject.

14. The tablet according to claim 13, wherein the maximum plasma concentration of strontium is from about 1 mg/L to about 8 mg/L.

15. The tablet according to claim 1, providing a steady state plasma concentration in a subject after repeated administration of the tablet to the subject through steady-state conditions, from about 5 mg/L to about 50 mg/L strontium.

16. The tablet according to claim 15, wherein the steady state plasma concentration is from about 8 mg/L to about 20 mg/L strontium.

17. The tablet according to claim 1 having an improved bioavailability of strontium compared with that of PROTELOS® sachets containing strontium ranelate when orally administered at the same dose of strontium.

18. The tablet according to claim 17, wherein the improved bioavailability expressed as AUC(tablet)/AUC(PROTELOS®, sachet) is larger than 1.

19. The tablet according to claim 18, wherein AUC(tablet)/AUC(PROTELOS®, sachet) is 1.05 or more.

20. The tablet according to claim 1 which is essentially bioequivalent with PROTELOS® when administered in a dose calculated as strontium that is at the about most about 90% w/w of the dose of strontium administered in the form of PROTELOS®.

21. The tablet according to claim 20, wherein the bioequivalence is determined by means of at least one of the following parameters: tₘₐₓ, cₘₐₓ, AUC₀₋ₜ, AUC_{0-infinity}, W₅₀, or W₇₅.

22. The tablet according to claim 1 further comprising a filler.

23. (canceled)

24. The tablet according to claim 22, wherein the filler is present in a concentration of from about 5% w/w to about 15% w/w.

25. The tablet according to claim 1 further comprising a binder.

26. The tablet according to claim 25, wherein the binder is a polymeric compound.

27. The tablet according to claim 26, wherein the polymeric compound is a polyvinylpyrrolidone.

28. The tablet according to claim 25, wherein the binder is present in a concentration of from about 1% w/w to about 8% w/w.

29. The tablet according to claim 1, further comprising one or more lubricant(s).

30. The tablet according to claim 29, wherein the lubricant is magnesium stearate, colloidal anhydrous silica, or a combination thereof.

31. The tablet according to claim 29, wherein the lubricant is present in a concentration from about 0.25% w/w to about 2.5% w/w

32. The tablet according to claim 1, having the following ingredients:
strontium malonate from about 75% w/w to about 90% w/w
filler from about 5% w/w to about 15% w/w
binder from about 1% w/w to about 8% w/w
lubricant(s) from about 0.5% w/w to about 5% w/w.

33. The tablet according to claim 32, having the following ingredients:
strontium malonate from about 75% w/w to about 90% w/w
microcrystalline cellulose from about 5% w/w to about 15% w/w
Polyvidone from about 1% w/w to about 5% w/w
Colloidal anhydrous silica from about 0.25% w/w to about 2.5% w/w Magnesium stearate from about 0.25% w/w to about 2.5% w/w.

34. The tablet according to claim 33, having the following ingredients:
strontium malonate about 83.3% w/w
microcrystalline cellulose about 12% w/w
Polyvidone about 3.3% w/w
Colloidal anhydrous silica about 0.7% w/w
Magnesium stearate about 0.7% w/w.

35. The tablet according to claim 1, wherein the amount of strontium malonate is from about 200 mg to 1800 mg.

36. The tablet according to claim 35, wherein the amount of strontium malonate is from 900 mg to 1200 mg.

37. The tablet according to claim 35 for once daily administration.

38. The tablet according to claim 35, wherein the amount of strontium malonate is from about 450 mg to about 600 mg.

39. The tablet according to claim 38, for twice daily administration.

40. The tablet according to claim 1, having the following ingredients:
strontium malonate about 83.3% w/w
microcrystalline cellulose about 12% w/w.

41. The tablet according to claim 2, containing 76% w/w or more of strontium malonate.

42. The tablet according to claim 2, containing 77% w/w or more of strontium malonate.

43. The tablet according to claim 2, containing 78% w/w or more of strontium malonate.

44. The tablet according to claim 2, containing 79% w/w or more of strontium malonate.

45. The tablet according to claim 2, containing 80% w/w or more of strontium malonate.

46. The tablet according to claim 2, containing 81% w/w or more of strontium malonate.

47. The tablet according to claim 2, containing 82% w/w or more of strontium malonate.

48. The tablet according to claim 2, containing 83% w/w or more of strontium malonate.

49. The tablet according to claim 2, containing 84% w/w or more of strontium malonate.

50. The tablet according to claim 2, containing 85% w/w or more of strontium malonate.

51. The tablet according to claim 5, wherein the aqueous medium is water.

52. The tablet according to claim 5, wherein the dissolution medium employed is a buffer solution having a pH in a range corresponding to pH 3-10.

53. The tablet according to claim 7, wherein at least 75% w/w is released within 30 min.

54. The tablet according to claim 7, wherein at least 80% w/w is released within 30 min.

55. The tablet according to claim 7, wherein at least 85% w/w is released within 30 min.

56. The tablet according to claim 7, wherein at least 90% w/w is released within 30 min.

57. The tablet according to claim 7, wherein at least 95% w/w is released within 30 min.

58. The tablet according to claim 19, wherein AUC(tablet)/AUC(PROTELOS®, sachet) is 1.1 or more.

59. The tablet according to claim 35, wherein the amount of strontium malonate is from about 300 mg to about 1500 mg.

60. The tablet according to claim 35, wherein the amount of strontium malonate is from about 500 mg to about 1500 mg.

61. The tablet according to claim 35, wherein the amount of strontium malonate is from about 600 mg to about 1200 mg.

62. The tablet according to claim 36, wherein the amount of strontium malonate is from about 1050 mg to about 1150 mg.

63. The tablet according to claim 38, wherein the amount of strontium malonate is from about 500 mg to about 600 mg.
